(19) United States
(12) Patent Application Publication
(10) Pub. No.: US 2001/0016747 A1
(43) Pub. Date: Aug. 23, 2001

(54) Method and apparatus for deploying a memory working tip member to or from an approach path to a desired working area

(76) Inventors: Jack W. Romano, Bellevue, WA (US); Kungle Chang, Livingston, NJ (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/732,512

(22) Filed: Dec. 7, 2000

Related U.S. Application Data
(63) Continuation of application No. PCT/US99/11484, filed on May 25, 1999.
(60) Provisional application No. 60/088,590, filed on Jun. 8, 1998.

(51) Int. Cl. A61B 17/04

(52) U.S. Cl. 606/148

(57) ABSTRACT

A wire loop (46) is carried at the distal end of a plunger (44 or 120) which, in turn, is telescoped inside a tubular chassis (40, 42). The proximate end of the plunger (44 or 120) projects from the tubular chassis (40, 42) and is connected to the end of a looped spring handle (60 or 60′) by means of which the plunger (44 or 120) can be reciprocated. At the distal end, a nose cone (52) is fitted in the tubular chassis (40, 42) and carries a guide conduit or tip (56) through which the wire loop (46) can be projected or retracted by corresponding movement of the plunger (40 or 120). The wire loop (46) is preformed to a desired shape and curvature and is formed of a material having memory characteristics such that it will return to the predetermined, precurved shape when it is allowed to relax by projection from the guide tip (56). Such loop (46) is capable of being forced to a straightened, compressed condition by retraction into the tip (56), which is achieved by manually withdrawing the plunger (44 or 120) by operation of the handle (60 or 60′). The handle (60 or 60′) can be provided with a spring latch (68, 70, 72) to retain the plunger (44 or 120) in a proximally slid condition with the wire loop (46) protruding, against the normal biasing force tending to retract the plunger (44 or 120) and wire loop (46).

### CROSS-REFERENCE TO RELATED APPLICATIONS

This application is a continuation of International Application No. PCT/US99/11484, filed May 25, 1999, which claims the benefit of priority of the filing date of U.S. Provisional Application No. 60/088,590, filed on Jun. 8, 1998.

### FIELD OF THE INVENTION

The present invention relates to a device having a tip member which can be manipulated to a working area where it may be used for delivering or retrieving an element, particularly a strand or cord element, and more particularly, a suture, such as during a surgical procedure.

### BACKGROUND OF THE INVENTION

A traditional suture passer consists of a long, rigid needle having its proximate end formed into or secured in a handle and a distal end with a hook or an eye for delivering or snaring a suture. More complicated suture passers have been developed with hook and/or eye members that move relative to another member, such as by being attached to a plunger that telescopes within a long, straight tube. In that case, the handle is provided with a slide, for example, that can be manipulated to move the plunger in and out. Nevertheless, known suture passers are not adapted for some complicated suture delivery/retrieval operations, such as delivery and retrieval of a suture through a curved bore in a bone. Such bores may approach 180 degrees in arcuate extent, and it can be difficult to thread suture through the hole, such as for attachment of torn or displaced body tissue in its anatomically correct location relative to the bone for a length of time sufficient that healing occurs and normal range of motion and strength returns.

### SUMMARY OF THE INVENTION

The present invention provides a system for deploying a working tip member, such as a wire loop, through a straight or curved tube, such as for delivering or retrieving a suture during a surgical procedure. In the preferred embodiment, the wire loop is carried at the distal end of a plunger which, in turn, is telescoped inside a tubular chassis. The proximate end of the plunger projects from the tubular chassis and is connected to the end of a looped spring handle by means of which the plunger can be reciprocated. At the distal end, a nose cone is fitted in the tubular chassis and carries a guide conduit or tip through which the wire loop can be projected or retracted by corresponding movement of the plunger. Registration members such as keys and keyways maintain the various components in a reliable rotational relationship to each other.

The wire loop member is preformed to a desired shape and curvature and is formed of a material having memory characteristics such that it will return to the predetermined, precurved shape when it is allowed to relax by projection from the guide tip. Such loop is capable of being forced to a straightened, compressed condition by retraction into the tip, which is achieved by manually withdrawing the plunger by operation of the handle. The handle can be provided with a spring latch to retain the plunger in a proximally slid condition with the wire loop protruding, against the normal biasing force tending to retract the plunger and wire loop.

The curvature and shape of the wire loop can be selected based on the intended application. For example, for use in retrieving a suture and withdrawing it through a curved bore in a bone, the curvature of the wire loop can be in excess of 180 degrees so that, with the guide tip positioned in or close to one opening into the bone, the loop can be projected into the curved bore and will naturally follow a curved path as it passes through the bore in deviation from the linear path of the plunger and the straight or curved exit passage of the guide tip. When the loop projects from the other end of the bore, the suture can be inserted through the wire loop, whereupon the suture can be threaded through the bore by retracting the wire loop back through the bore and into the tip.

Other more detailed aspects of the invention are described below.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a top front perspective of an apparatus in accordance with the present invention, with parts shown in exploded relationship, such parts including a chassis-handle component, plunger component, delivery/retrieving wire component, and guide tip component;

FIG. 2 is a top rear perspective of the chassis component of the apparatus of FIG. 1;

FIG. 3 is a top plan thereof;

FIG. 4 is a side elevation thereof;

FIG. 5 is a section along line 5-5 of FIG. 4;

FIG. 6 is a section along line 6-6 of FIG. 3;

FIG. 7 is an enlarged detail section of the leading and upper end portion of the chassis component;

FIG. 8 is an enlarged detail section of the rear and upper end portion thereof;

FIG. 9 is an enlarged detail section of the part indicated by circle 9 of FIG. 7;

FIG. 10 (on the drawing sheet with FIG. 6) is a front elevation thereof;

FIG. 11 is an enlarged end elevation of the part indicated by circle 11 of FIG. 10;

FIG. 12 is a top front perspective of the plunger component of the apparatus of FIG. 1;

FIG. 13 is a top plan thereof;

FIG. 14 is a side elevation thereof;

FIG. 15 is a section along line 15-15 of FIG. 13;

FIG. 16 is an enlarged section of the area indicated by circle 16 of FIG. 15;

FIG. 17 is an end elevation thereof taken from the left of FIG. 14;

FIG. 18 is a top plan of the delivery/retrieving wire component of the apparatus of FIG. 1 with the wire in a straightened condition;

FIG. 19 is a side elevation thereof with the wire in its relaxed precurved and preshaped condition;

FIG. 20 is a top, rear, right perspective of a first part of the guide tip component of the apparatus of FIG. 1;

FIG. 20A is a top, rear, left perspective thereof;

FIG. 21 is a right side elevation thereof;

FIG. 22 is a section along line 22-22 of FIG. 21;

FIG. 23 is an end elevation taken from the left of FIG. 21;

FIG. 24 is an end elevation taken from the right of FIG. 21;

FIG. 25 is a left side elevation thereof;

FIG. 26 is a top rear perspective of a second part of the guide tip component of the apparatus of FIG. 1;

FIG. 27 is a side elevation thereof;

FIG. 28 is a vertical axial section thereof;

FIG. 29 is an enlarged sectional view of the part indicated by circle 29 of FIG. 28;

FIGS. 30 and 31 are corresponding side elevations of the apparatus of FIG. 1 with parts assembled and parts in different positions;

FIG. 32 is a diagrammatic side elevation of an alternative embodiment of an apparatus in accordance with the present invention; and

FIG. 33 is a corresponding side elevation with parts in different positions.

### DETAILED DESCRIPTION OF THE PREFERRED EMBODIMENT

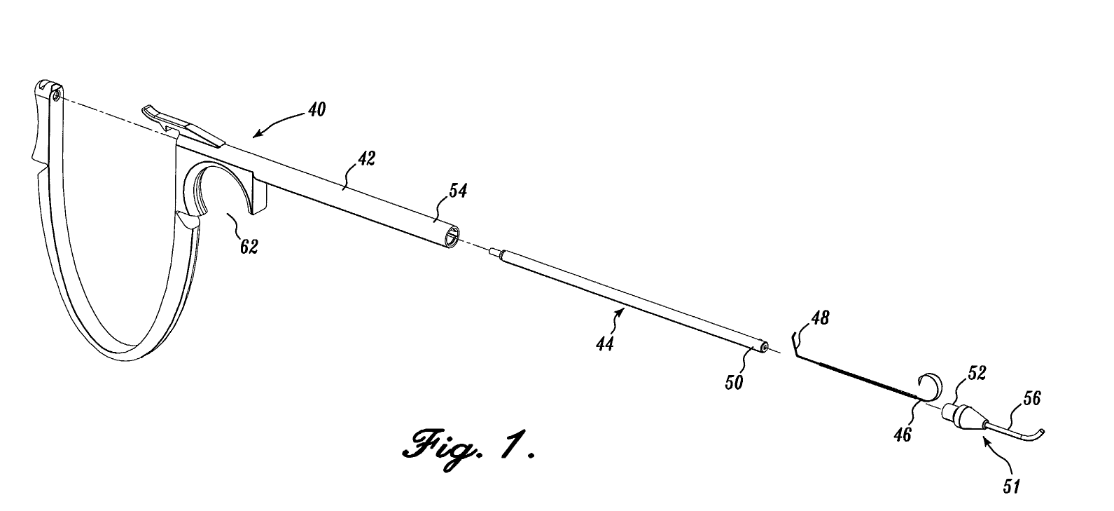

With reference to FIG. 1, a preferred embodiment of an apparatus for deploying a memory working tip member to or from an approach path to a desired working area in accordance with the present invention includes a chassis 40 including a front or distal hollow barrel portion 42. A plunger component 44 is slidable inside the barrel portion. A delivery/retrieving wire loop 46 has a rear end 48 secured to the leading end 50 of the plunger. A guide tip component 51 has a rear stem 52 fittable in the leading end portion 54 of the chassis barrel 42, and a curved conduit section 56 projecting forward or distally from the stem. In the preferred illustrated embodiment, the apparatus is configured as a suture passer, which is the application described in detail below. It should be understood, however, that other applications are possible, such as delivery/retrieving flexible strands, cords, cables or other elements in hard to reach areas, particularly when it is desirable to wrap, thread or otherwise move such an element around or through an obstruction with the angle of delivery or retrieval approaching or exceeding 180 degrees.

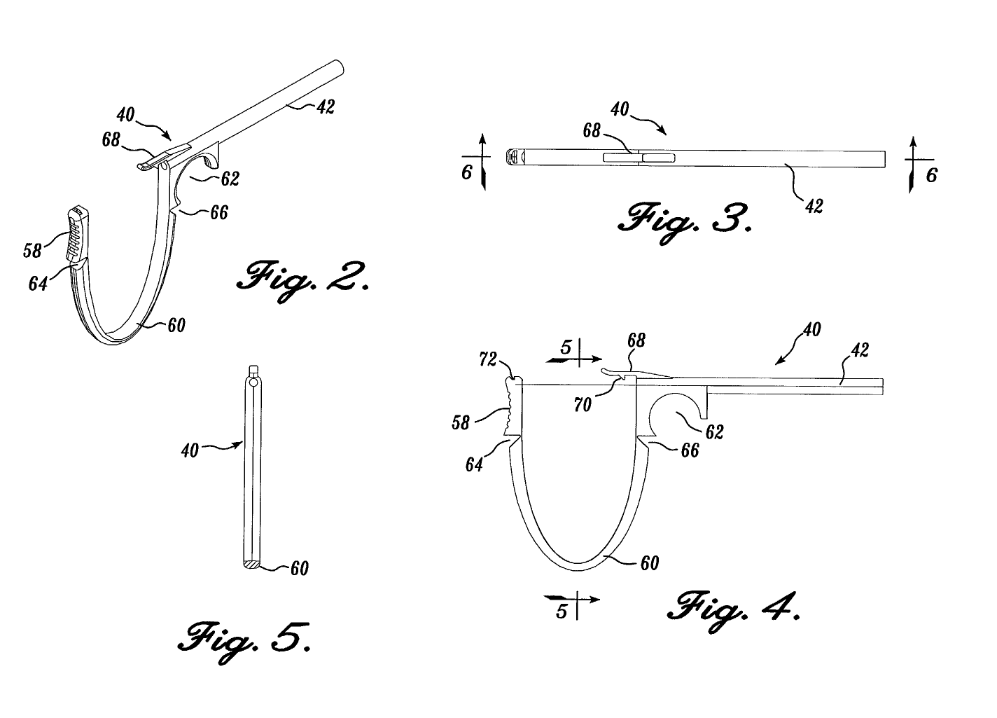

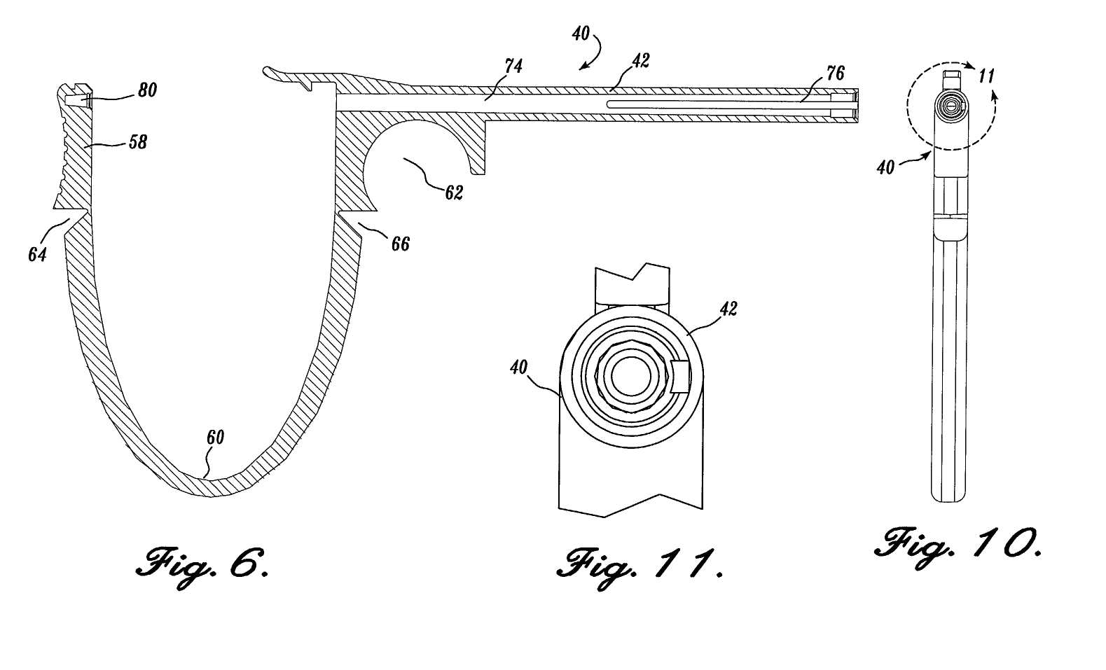

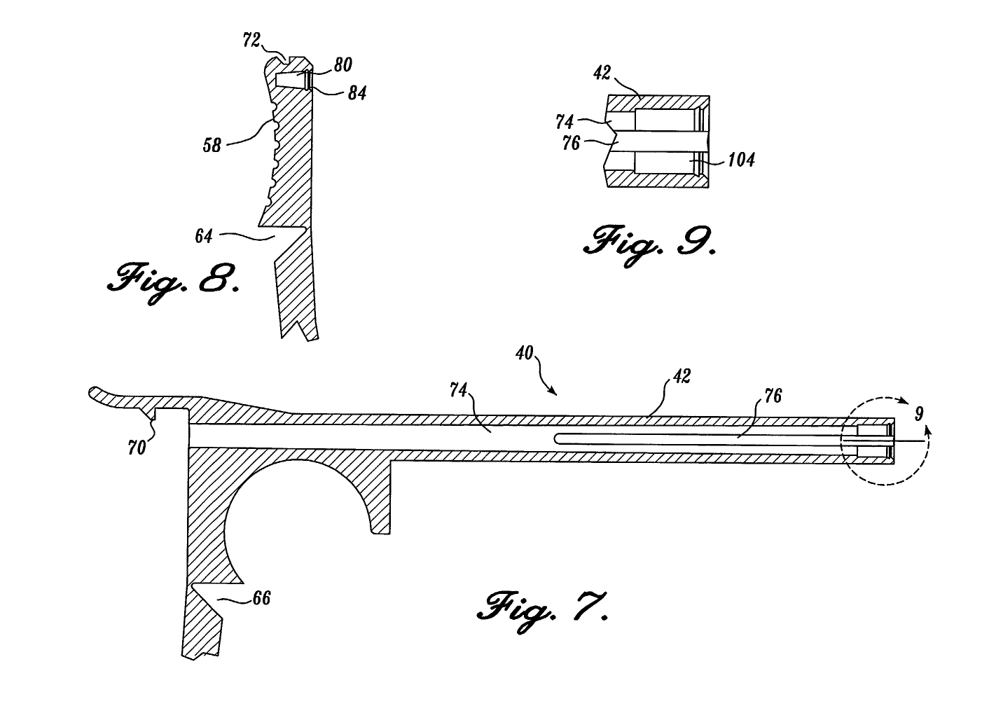

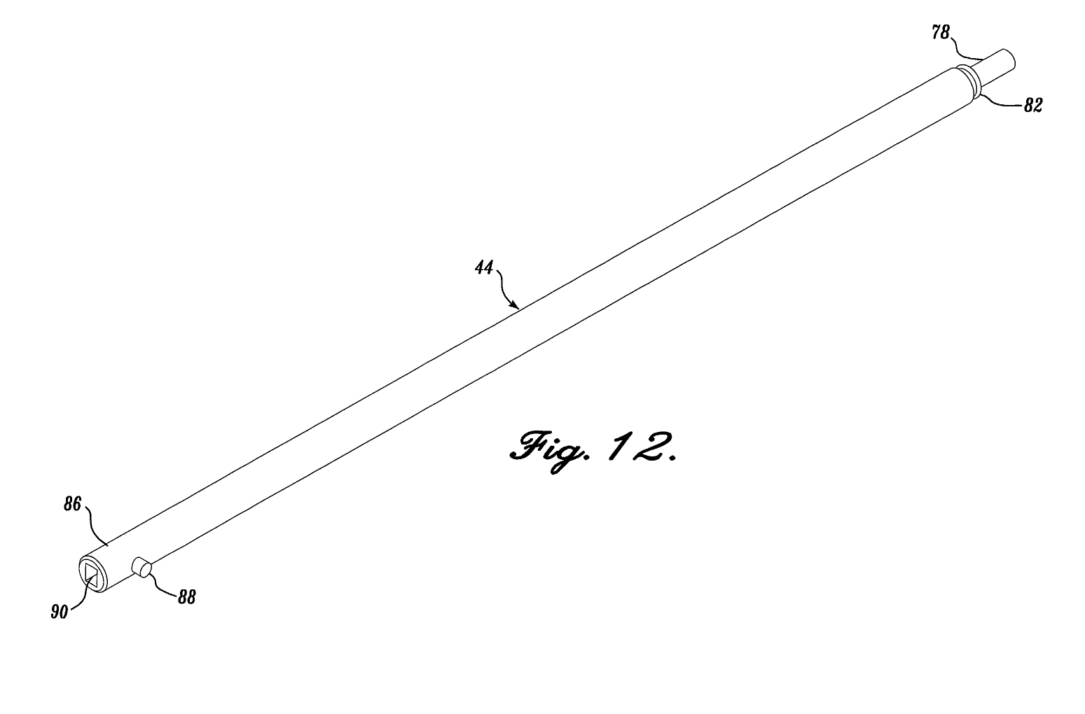

Details of the chassis 40 are shown in FIGS. 2-11. Beginning from the rear or proximate end 58 of the chassis (FIG. 2), an arcuate handle loop 60 leads to a finger notch 62 located at the rear of and underneath the barrel 42. The finger notch could be formed as a closed loop, and an additional notch or loop provided at the opposite side of the barrel for two-finger stability. The handle loop 60 is notched at a first location 64 close to its rear end and at a second location 66 close to its front end. At the top of the rear end portion of the barrel, a releasable latch 68 projects rearward, with a downward extending tooth 70 (FIG. 4, for example) engageable in an upper notch 72 of the rear end portion of the handle when the rear end portion is “squeezed” forward, as described below in connection with operation of the apparatus. The barrel 42 has an axial through bore 74 (FIG. 7) with a keyway 76 along one lateral side.

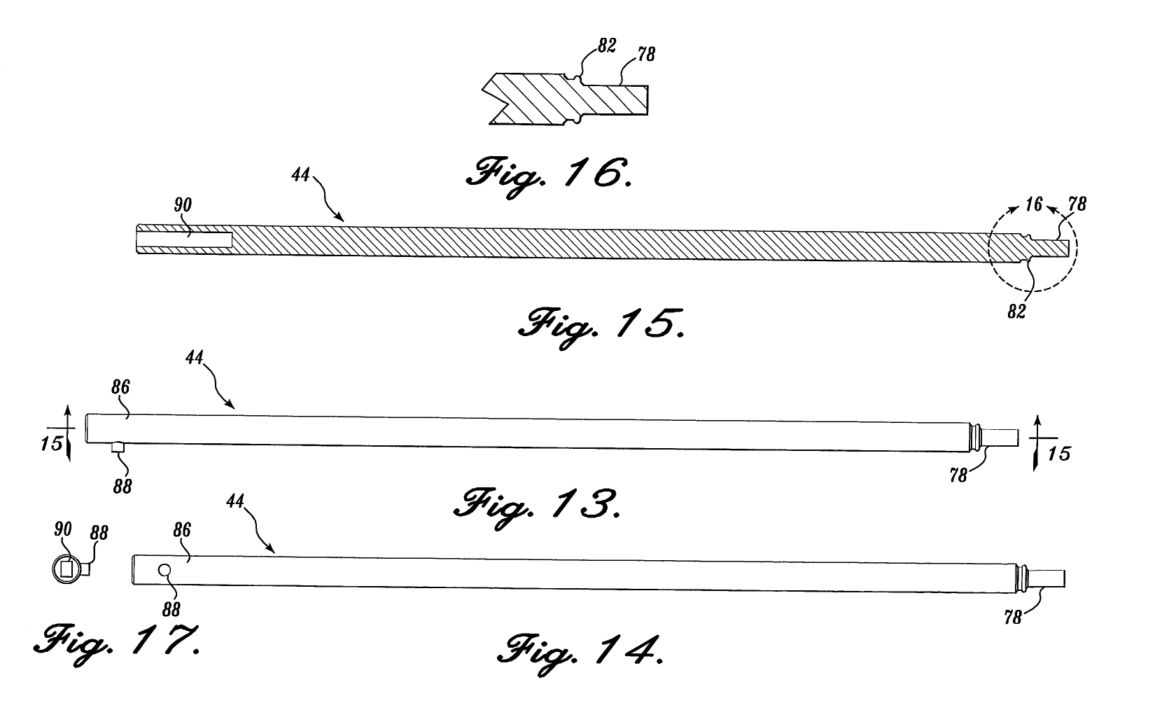

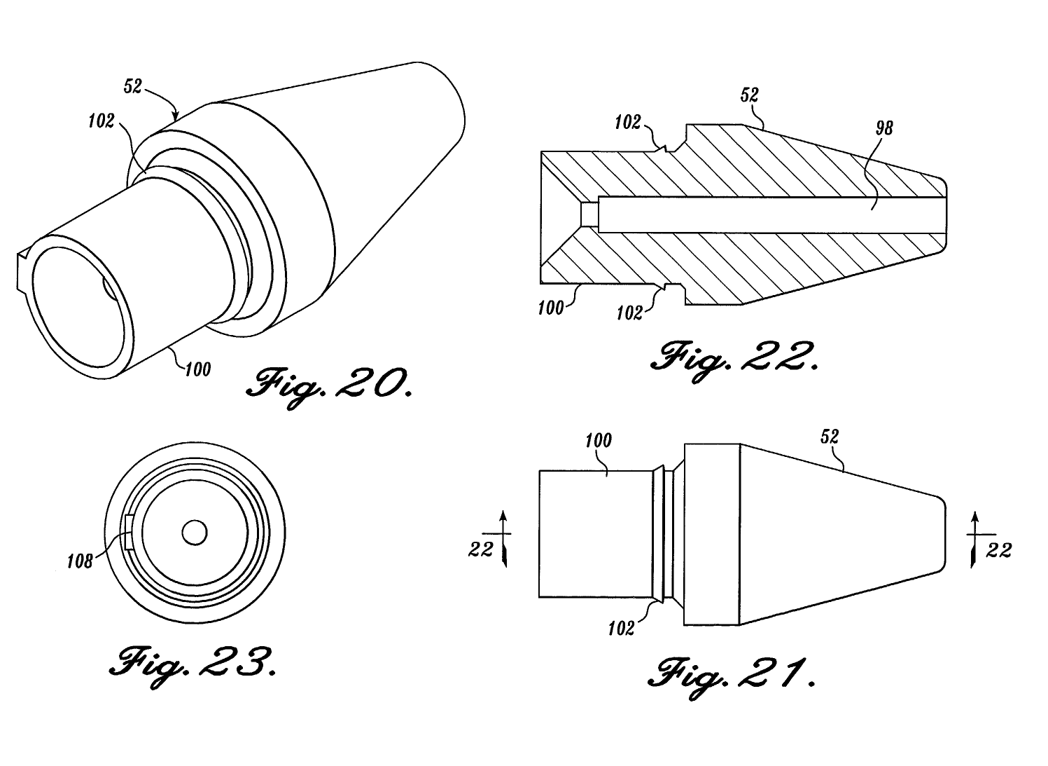

The plunger component 44 is shown in FIGS. 12-17. For the major portion of its length, the plunger component is of uniform circular cross section, slightly smaller than the diameter of the bore through the barrel of the chassis for a smooth sliding fit. The rear end portion 78 is a short stub or stem of reduced diameter for fitting in a tapered socket 80 of the chassis handle (see FIG. 8). The stem is formed with a circumferential rib 82 (FIG. 16) for a snap fit in a circumferential groove 84 (FIG. 8) of the socket 80. Adhesive may be used to secure the stub or stem in the socket 80 if it is not desired to disassemble the parts, such as for separate sterilization, cleaning, or replacement.

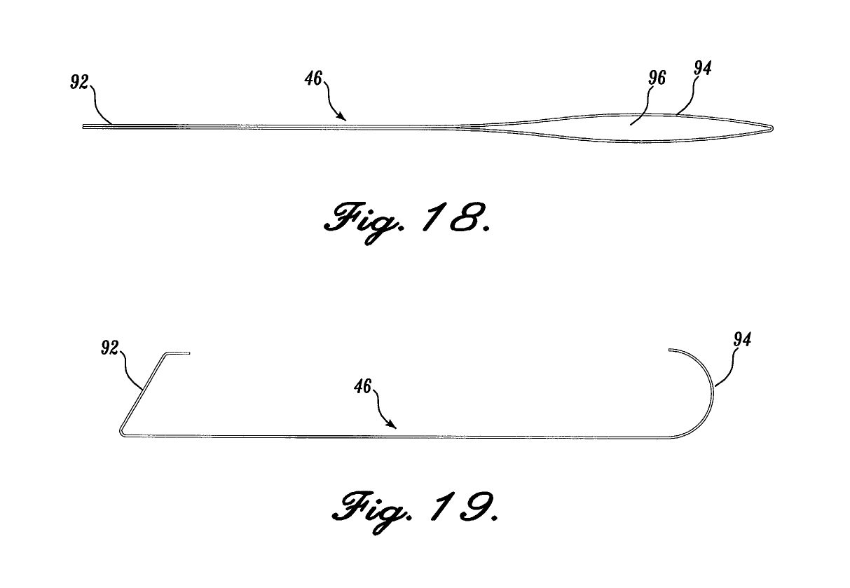

The leading end 86 of the plunger component has a transversely extending projection 88 for riding in the keyway 76 seen in FIG. 7. This maintains the same rotational relationship of the plunger to the barrel as the plunger is moved fore and aft. A blind socket 90 (FIGS. 12 and 15) extends axially inward from the leading end of the plunger.

The delivery/retrieving wire component 46 (sometimes referred to as the memory working tip member) is shown in FIGS. 18 and 19. Such wire component is preferably formed of a single strand of resilient wire precurved to a desired shape and having memory characteristics such that the wire returns to a predetermined shape and curvature when relaxed, despite being capable of being forced to a straightened, compressed condition. The rear end portion 92 of the wire fits in the socket 90 (FIGS. 12 and 15) of the plunger component for a secure connection of the two. The forward portion 94 of the wire is curved through an angle of approximately 180° as seen in FIG. 19, and, when relaxed, forms an open loop with a central aperture or eyelet 96 as shown in FIG. 18. The degree of curvature and shape of the loop will depend on the intended application, i.e., the desired position of the loop when projected and shape required to deliver or retrieve the desired element.

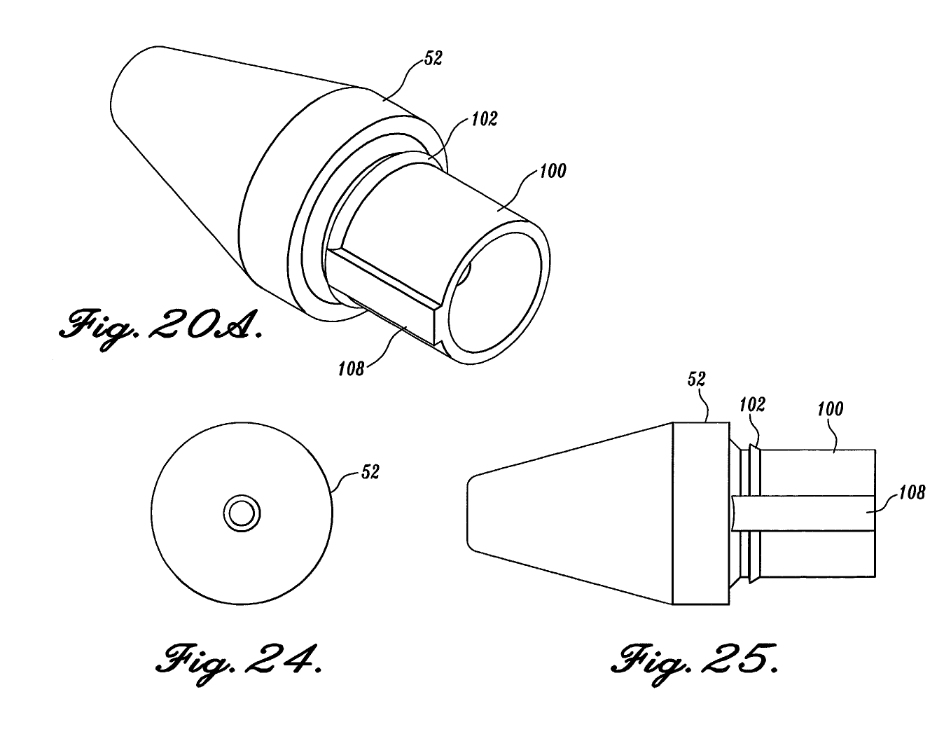

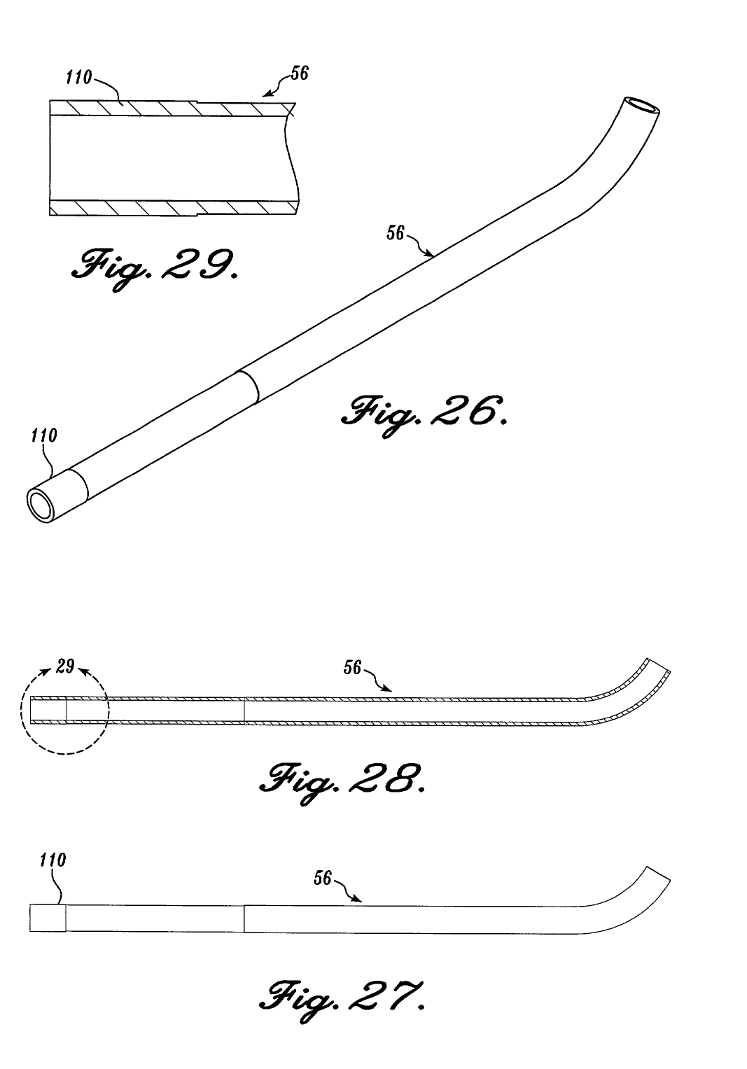

The rear part 52 of the guide tip component 50 of the suture passer in accordance with the present invention is shown in FIGS. 20-25. It includes an axial through bore 98 (FIG. 22) and a rear stem 100 with a circumferential rib 102 for fitting in a front socket portion 104 (FIG. 9) of the leading end of the chassis component barrel 42. Stem 100 also has a side projection 108 (FIGS. 23 and 25) for fitting in the keyway 76 (FIG. 7) to orient the stem.

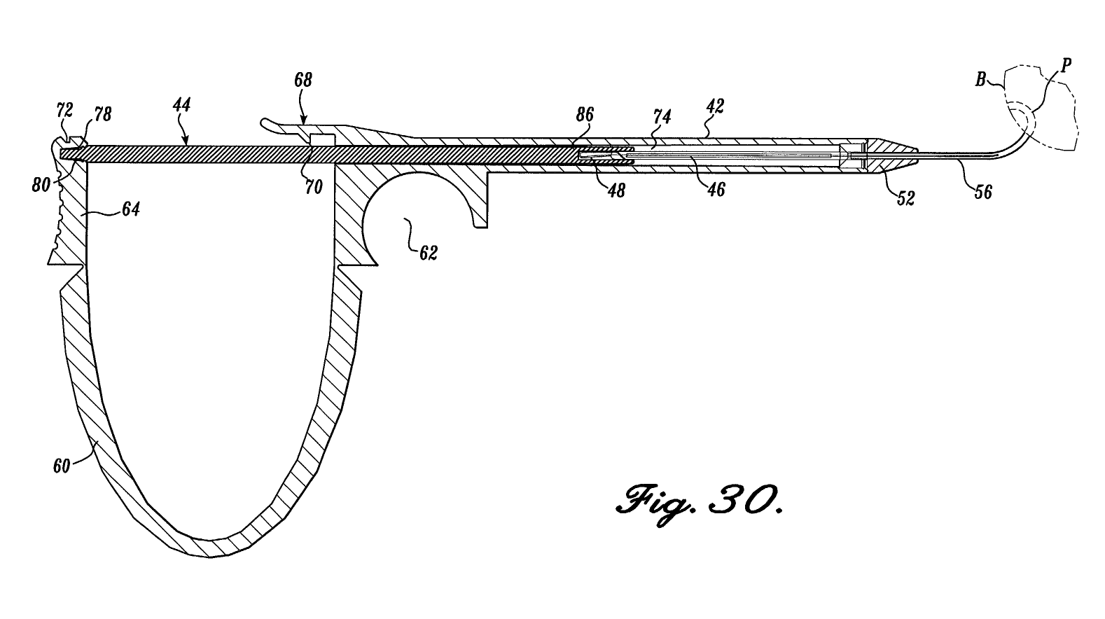

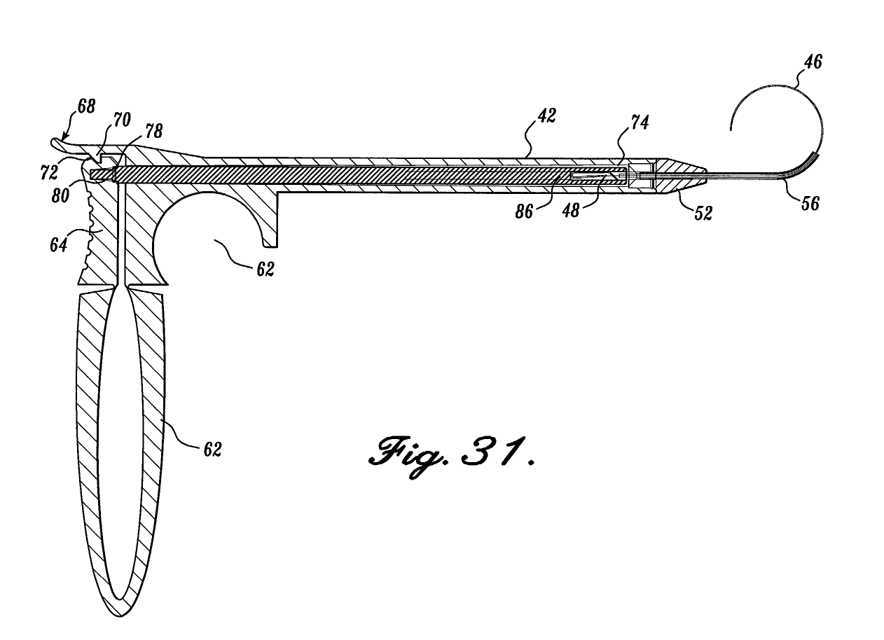

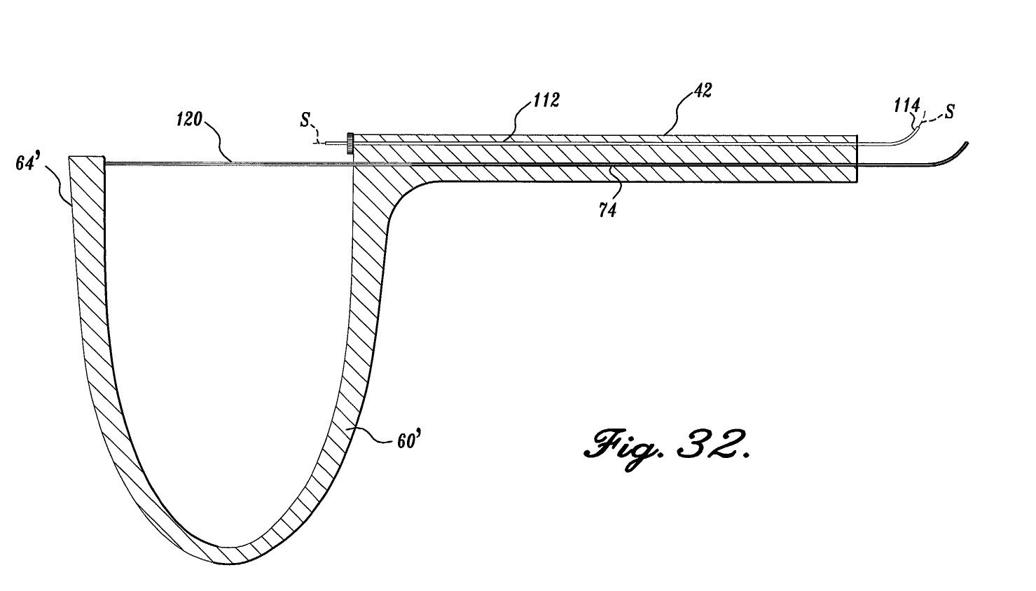

The conduit section 56 of the guide tip component is shown in FIGS. 26-29. It includes a rear stem portion 110 for a tight press fit in the bore 98 (FIG. 22) of stem section 52. From such stem section 110 the tip section continues linearly forward and then is curved gently upward. The joining of the conduit section in the stem section preferably is sufficiently tight so as to prevent rotation of the conduit section relative to the stem.

The chassis component, including handle and latch, plunger, and guide tip all can be formed of suitable plastic such as polypropylene. As noted above, the wire must be precurved with memory characteristics allowing it to be straightened, but which will return to the precurved condition when relaxed. A nickel-titanium alloy of a diameter of 0.010 inch is an appropriate material for the wire.

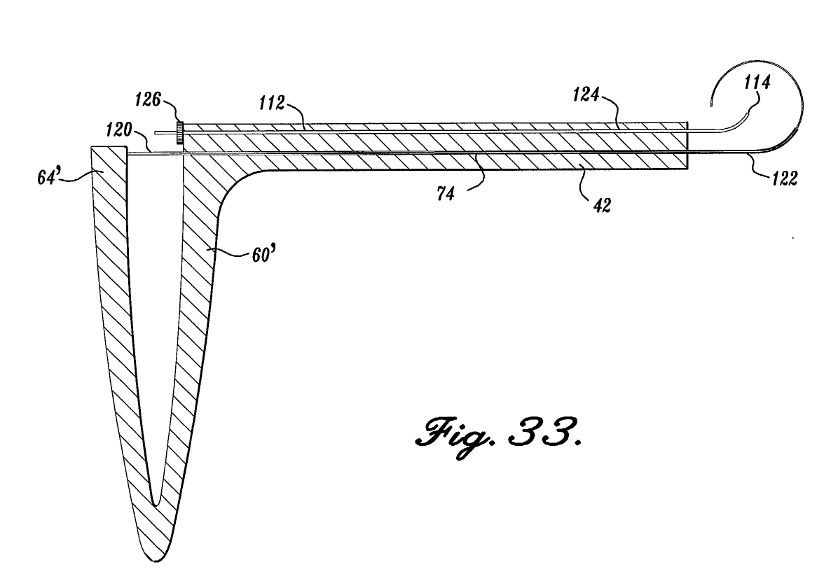

FIG. 30 shows the parts of the suture passer in accordance with the present invention in assembled condition. The rear end 78 of the plunger 44 is secured in the socket 80 of the chassis handle 60. The forward end 86 of the plunger is slidably received in the bore 74 of the chassis barrel 42. The suture retrieving wire 46 has its rear end portion 48 joined to the leading end portion 86 of the plunger, and extends forward through the rear part 52 and into the wire guiding conduit 56. In this condition (the relaxed condition of the handle 60), the wire is straight and extends approximately to the location where the wire guiding conduit 56 starts to curve. The suture retrieving wire 46 can be projected from the conduit by squeezing the handle section 60, bringing its rear end 64 into close proximity with the finger notch 62 of the chassis component. In this condition, the tooth 70 of the latch 68 snaps into the groove 72 of the rear portion 64 of the handle 60 to retain the parts in position, as seen in FIG. 31. Projection of the wire allows it to assume its precurved shape as it is extended, including expansion near the distal tip of the wire to form the eyelet. This procedure can be used for feeding a suture through bone or body tissue, particularly through a curved passage in a bone which may be formed by a special instrument for drilling a curved bore. For example, in FIG. 30 a bone B is shown diagrammatically, including a curved passage P. The tip of the conduit 56 of the instrument can be placed close to or slightly inside the passage P, followed by projection of the wire which gradually feeds first away from the guide tip, and through the curved passage to an intermediate position from which the distal end of the wire curves back toward the guide tip and where the eyelet at the end projects from the other end of the passage. From that location a suture can be fed into the eyelet, the latch released, and the wire withdrawn toward the chassis by the natural spring force of the handle.

The latch provided need not have a single latching position, but rather can be a ratcheting type of latch so that the wire can be retained in a plurality of different projected positions, depending on the application. With reference to FIG. 32, instead of providing a separate plunger extending through the barrel of the chassis, a longer wire can extend the full length of the barrel and be connected to the rear portion 64′ of the handle, or other mechanism for reciprocating the wire lengthwise of the barrel can be provided. Another modification of the embodiment of FIG. 32 is the provision of an additional bore 112 immediately above the wire receiving bore 74 for feeding a suture S or other flexible element through the barrel 42. A curved tip 114 can be provided to direct the suture to a desired position where it can be fed directly through the eyelet of the wire loop when the wire is projected. See, for example, FIG. 33.

With reference to FIG. 32 and FIG. 33, the proximate or rear end portion of the delivery/retrieving wire can be encased in a narrow sheath 120 to reinforce the wire, particularly the section bridging between the rear and forward portions of the handle 60′. The rear end portion of the sheath and wire can be connected to the rear portion 64′ of the handle by any suitable connection. The wire can be guided through a long tube 122 extending the full length of the barrel 42 of the chassis and including the upwardly curved distal tip section, or the distal tip section can extend only a short distance into the bore 74 through the barrel. For guiding the suture, an upper tube 124 can be provided, extending the full length of the chassis barrel 42 and having the upwardly curved distal tip portion 114. In the illustrated embodiment, tube 124 is slidably and rotatably received in a bore through the chassis handle, for manipulating the tip section 114 fore and aft and rotating it as required to project the suture or other flexible element through the eyelet of the suture retrieving wire. For this purpose, an enlarged end 126 can be provided on the suture receiving tube 124 at its trailing end portion to allow a convenient mechanism for manipulating the tube.

FIGS. 32 and 33 show the separate bores or lumens 74, 112 formed in a single integral chassis. A first alternative is to provide separate tubes that are held together by brace supports, or separate tubings that are insert-molded into a structural support. The lumens could be longitudinal cavities which are fitted at the distal ends with separate, press-fit distal tubing ends having the curved tip portions, or the shorter tip portions could be adhesively secured in the end of the chassis. The size and shape of the tubing tips would correspond to the specific size of the memory precurve of the wire delivery/retrieving member, which may correspond to the radius of a curved tunnel in a bone, and the desired angle of deflection required by the suture to feed into the memory loop. As with the previously described embodiment, it should be noted that the delivery/retrieving wire loop first projects outward, away from the distal end of the guide tube, then rearward back toward the guide tube where it will be intersected by a suture or other flexible element fed through the upper curved guide.

The apparatus can be designed for easy assembly and disassembly to allow cleaning and sterilization of the individual pieces. For example, in the embodiment shown in FIGS. 1-31, the proximate end 48 of the delivery/retrieval wire loop member is inserted into the socketed end of the plunger 44. Plunger 44 is slid into the bore of the chassis barrel 42, and has its distal end or stem 78 press- or snap-fitted into the shallow socket 80 of the rear end portion of the handle loop 60. The guide tip 51, including the distal conduit 56, can have the wire loop threaded through them and be snugly inserted into the distal opening of the chassis barrel 42, with the side key 108 of the tip received in the keyway 76. Disassembly is achieved by the reverse procedure, whereupon the lumen or lumens of the housing structure can be cleaned by forced air or an appropriate cleaning liquid and/or by an ultrasonic cleaner. A small pipe cleaner can be used as necessary, the goal being that smooth, consistent operation is achieved following sterilization and re-assembly of the device.

While the preferred embodiment has been disclosed, it should be understood that various changes can be made without departing from the spirit of the invention, which includes the provision of a precurved memory tip member from a straightened condition to the precurved “relaxed” condition, preferably forming an eyelet or other retrieval mechanism, to capture a suture or other flexible member and allow it to be moved to a desired position such as back through a curved bore of a bone or through or around other tissue or an obstruction.

The embodiments of the invention in which an exclusive property or privilege is claimed are defined as follows:

1. Apparatus for delivery/retrieval of a flexible element comprising:
a working tip member formed of a resilient material having memory characteristics, the working tip member having a first predetermined shape in a relaxed configuration adapted for receiving the flexible element; and
a tubular delivery member including a guide section for receiving the working tip member for sliding therealong between a projected position in which the working tip member is in the relaxed configuration and in position for receiving/delivering the flexible member and a retracted position in which the working tip member is guided to a second configuration different from the relaxed configuration, the second configuration being determined by the shape of the guide section of the delivery member, the delivery member including a plunger slidable in the tubular guide section and adapted for carrying the working tip member for movement therewith, and the plunger and tubular guide section have a cooperating key and keyway for maintaining a desired orientation of the plunger relative to the tubular guide section.

2. The apparatus defined in claim 1, in which the working tip member includes a length of resilient wire.

3. The apparatus defined in claim 2, in which the wire is precurved to assume an arc of predetermined angular extent when the working tip member is in the projected position.

4. The apparatus defined in claim 3, in which the delivery member defines a delivery path therethough, the precurved wire deviating from the delivery path as the precurved wire is moved to the projected position.

5. The apparatus defined in claim 4, in which the delivery path includes a linear portion.

6. The apparatus defined in claim 5, in which the delivery path includes a proximate linear portion and a distal curved exit portion, the precurved wire extending from the exit portion in the projected position of the working tip member.

7. The apparatus defined in preceding claim in which the working tip member defines a loop in the projected position for receiving the flexible element.

8. The apparatus defined in claim 7, in which the opening of the loop is of a greater cross-sectional size when the working tip member is in the projected position than when the working tip member is in the retracted position.

9. The apparatus defined in claim 1, in which the working tip member is precurved through an angle of greater than 90 degrees in the projected position.

10. The apparatus defined claim 9, in which, in the retracted position of the working tip member, the working tip member is generally linear.

11. The apparatus defined in claim 1, in which the working tip member is precurved through an angle of greater than 180 degrees in the projected position, and in which the working tip member is restrained to an angular extent much less than 180 degrees in the retracted position.

12. The apparatus defined in claim 1, in which the delivery member includes a manually controlled actuator for moving the plunger in a linear path.

13. The apparatus defined in claim 12, in which the actuator includes a resilient handle loop having an end portion connected to a proximate end of the plunger.

14. The apparatus defined in claim 13, in which the delivery member includes a latch for releasably locking the actuator in a position in which the working tip member is in its relaxed configuration projecting from the delivery member guide section.

15. The apparatus defined in claim 1, in which the delivery member guide section includes a nose cone mounted in the distal end of the tubular guide section and having an internal passage therethrough.

16. The apparatus defined in claim 15, in which the nose cone is keyed to the tubular guide section for maintaining a desired rotational relationship between the two.

17. The apparatus defined in claim 15, in which the guide section includes an exit tube secured in the nose cone and forming a smooth continuous passage with the internal passage of the nose cone.

18. The apparatus defined in claim 17, in which the exit tube is curved through a predetermined angle.

19. The apparatus defined in claim 18, in which the predetermined angle is less than 90 degrees.

20. The apparatus defined in claim 1, in which the delivery member defines a guide passage therealong, the working tip member being precurved through a predetermined angle for deviating from the guide passage as it is moved from the retracted position to the projected position.

21. The apparatus defined in claim 20, in which the angle of deviation is at least 90 degrees so that the working tip member moves first outward away from the delivery member then transversely of the length guide passage as the working tip member is moved from the retracted position to the projected position.

22. The apparatus defined in claim 20, in which the working tip member is precurved through an angle of at least about 180 degrees so that the working tip member moves first outward in the general distal direction of the delivery path, then transversely of the path, then in a direction generally proximally of the delivery path adjacent to the exit end of the delivery member as the guide tip member is moved from the retracted position to the projected position.

23. The apparatus defined in claim 1, in which the working tip member is precurved for deviating in a direction around or through an object for transportation of the flexible element to or from a desired working space separated by the object from the delivery member.

24. The apparatus defined in claim 1, in which the working tip member is preformed so as to project initially away from (distally from) then generally back toward (proximally toward) the delivery member as the working tip member is moved from the retracted position to the projected position, without altering the position of the delivery member.

25. Apparatus for delivery/retrieval of a flexible element comprising:
a working tip member formed of a resilient material having memory characteristics, the working tip member having a first predetermined shape in a relaxed configuration adapted for receiving the flexible element; and
a tubular delivery member including a guide section for receiving the working tip member for sliding therealong between a projected position in which the working tip member is in the relaxed configuration and in position for receiving/delivering the flexible member and a retracted position in which the working tip member is guided to a second configuration different from the relaxed configuration, the second configuration being determined by the shape of the guide section of the delivery member, the delivery member guide section including a nose cone mounted in the distal end of the tubular guide section and having an internal passage therethrough.

26. The apparatus defined in claim 25, in which the nose cone is keyed to the tubular guide section for maintaining a desired rotational relationship between the two.

27. The apparatus defined in claim 25, in which the guide section includes an exit tube secured in the nose cone and forming a smooth continuous passage with the internal passage of the nose cone.

28. The apparatus defined in claim 27, in which the exit tube is curved through a predetermined angle.

29. The apparatus defined in claim 28, in which the predetermined angle is less than 90 degrees.
